(19) United States
(12) Patent Application Publication
(10) Pub. No.: US 2025/0127609 A1
(43) Pub. Date: Apr. 24, 2025

(54) Apparatus for Injecting Implant for Eye Disease

(71) Applicant: MICROT INC., Seoul (KR)

(72) Inventors: Junoh Ryu, Gwacheon (KR); Doohwan Kwak, Yongin (KR); Jiyeon Kim, Seoul (KR)

(73) Assignee: MICROT INC., Seoul (KR)

(21) Appl. No.: 18/260,809

(22) PCT Filed: Dec. 2, 2022
(86) PCT No.: PCT/KR2022/019498

§ 371 Date: Jul. 10, 2023

(30) Foreign Application Priority Data
Jul. 12, 2022 (KR) 10-2022-0085392

(51) Int. Cl. A61F 2/14; A61B 17/34

(52) U.S. Cl. CPC A61F 2/148; A61B 2017/3427

(57) ABSTRACT

An apparatus for injecting an implant for eye disease to regulate the intraocular pressure includes a cannula having an internal space. The cannula includes a first segment including one end; a second segment connected to the first segment; and a third segment connected to the second segment, and including an opening for the implant on a side. The opening for the implant is spaced a predetermined distance apart from one end of the cannula to provide a structure for allowing the insertion of only part of the implant and easy separation of the cannula and the implant in the eye.

### TECHNICAL FIELD

Embodiments relate to an apparatus for injecting an implant for eye disease, and more particularly, to an apparatus for easily injecting an implant into an eye with minimized damage to the eye in a method for lowering the intraocular pressure by the drainage of aqueous humor through the tube-shaped implant injected into the eye.

### BACKGROUND ART

When intraocular pressure lowering drugs are not effective in regulating the intraocular pressure in some patients with glaucoma, surgical treatments are performed to create pathways for the drainage of aqueous humor from the anterior chamber of the eye below the conjunctiva outside of the eye in order to lower the intraocular pressure. Trabeculectomy, a type of glaucoma filtration surgery for creating pathways or fistulas for aqueous humor drainage, is performed when the pathways are blocked again after surgery and has the reduced drainage of aqueous humor, failing to modulate the intraocular pressure. When the glaucoma filtration surgery is performed again due to the failed surgery, obstruction of aqueous humor drainage occurs more frequently after surgery and the surgery success rate is low.

Additionally, in the case of so-called inveterate glaucoma such as neovascular glaucoma or secondary glaucoma caused by uveitis according to the type of glaucoma, trabeculectomy has undesirable outcomes, such as frequent blockage of pathways after surgery. In the case of eyes with failed glaucoma filtration surgery or inveterate glaucoma, surgery for placing glaucoma implant devices (glaucoma implant surgery) is performed to prevent the blockage of pathways to increase the surgery success rate. Since the glaucoma implants effectively lower the intraocular pressure and provide the predictable clinical outcomes after surgery according to the preset inner diameter of the tube, the glaucoma implants have been used as an alternative to trabeculectomy especially in many types of glaucoma difficult to treat.

However, the existing glaucoma implants used in glaucoma implant surgery may cause many problems and complications such as difficulties in surgery due to the relatively large size, exposure and infections after surgery, eye movement disorders caused by the large body and consequential double vision. Accordingly, recently, Minimally-Invasive Glaucoma Surgery (MIGS) using small-sized glaucoma implant tools has been developed to lower the intraocular pressure using glaucoma implants more easily and reduce postoperative adverse effects caused by the large size.

The surgery using the small-sized glaucoma implants only can be conducted by injecting the small-sized glaucoma implant into the anterior chamber of the eye under the conjunctiva. However, for the drainage of aqueous humor from the eye under the proper pressure, it is necessary to inject and fix the glaucoma implant having a very small size to a proper position in the eye.

However, the existing apparatus for injecting an implant for eye disease is injected into the eye with the implant itself inserted into a cannula of the injection apparatus, and the implant is pushed and injected into the eye. This method causes damage to the eye surface tissue as much as the outer diameter of the injection apparatus that is larger than the outer diameter of the implant, and when the implant has a protruding structure (for example, a wing or an arm) in the lateral direction, not in the length direction, the injection apparatus needs to have the inner diameter that is large enough to cover the whole, resulting in the increased cross area of the injection region of the injection apparatus.

Additionally, the implant for eye disease often fails to perform the drainage function due to fibrosis after surgery, and in this case, additional surgery is required, and the injection apparatus configured to surround the implant increases the damage range of the eye surface tissue, resulting in higher risks of infections from external sources.

To solve this problem, there is a need for a method and apparatus for injecting an implant with minimized damage range of the eye surface tissue.

### RELATED LITERATURES

(Patent Literature 001) U.S. Pat. No. 9,649,223 B2

### DISCLOSURE

### Technical Problem

The present disclosure is designed to solve the above-described problems, and therefore the present disclosure is directed to providing an apparatus for injecting an implant for eye disease with minimized eye tissue damage range in which a cannula of the apparatus for injecting an implant for eye disease has an opening for the implant through which part of the implant is injected and fixed when injecting the implant into the eye.

### Technical Solution

An apparatus for injecting an implant for eye disease according to an embodiment of the present disclosure includes a cannula having an internal space, wherein the cannula includes a first segment including an end of the cannula; a second segment connected to the first segment; and a third segment connected to the second segment, and including an opening for the implant on a side.

In an embodiment, a length of the second segment may be equal to or larger than 1 mm.

In an embodiment, a sum of lengths of the first segment and the second segment may be equal to or smaller than 10 mm.

In an embodiment, the opening for the implant may have different heights of a first open end and a second open end, the first open end may be adjacent to the second segment, and the second open end may be opposite the first open end.

In an embodiment, each of the first open end and the second open end may have any one shape of a sloped surface inclined inward the cannula, an inclined curved surface, a vertically curved surface and a vertically sloped surface.

In an embodiment, angles at which the first open end and the second open end are inclined inward the cannula may be different from each other.

In an embodiment, the height of the first open end may be larger than the height of the second open end.

In an embodiment, the end of the cannula may be open.

In an embodiment, the second segment may include a protrusion disposed adjacent to the opening for the implant.

In an embodiment, the apparatus for injecting an implant for eye disease may further include the implant for eye disease, wherein a body of the implant for eye disease may be tubular in shape, and at least part of the implant for eye disease may be disposed in the second segment.

In an embodiment, part of the implant for eye disease may be inserted into the cannula toward the first segment through the opening for the implant.

In an embodiment, the implant for eye disease may include a core therein.

In an embodiment, the implant for eye disease may include a wing protruding in a direction that is different from a lengthwise direction.

In an embodiment, the implant for eye disease may further include a matrix which covers the implant at least in part.

In an embodiment, the matrix may be impregnated or impregnable with a solution for eye disease.

### Advantageous Effects

According to the an apparatus for injecting an implant for eye disease in accordance with an embodiment of the present disclosure, it is possible to minimize the eye tissue damage range when injecting the implant for eye disease into the anterior chamber of the eye, and keep the size of the internal structure of the injection apparatus small since part of the implant that is not injected into the anterior chamber is not inserted into the injection apparatus and is disposed outside of the injection apparatus.

Additionally, since the implant is injected into the eye with only part of the implant inserted into the injection apparatus, it is possible to universally use the injection method or the structure of the injection apparatus even when the implant has a structure that protrudes to the side such as a wing, an arm and a matrix at the rear end.

### DESCRIPTION OF DRAWINGS

FIGS. 1 and 2 show an apparatus 1000 for injecting an implant for eye disease according to an embodiment of the present disclosure.

FIG. 3 shows a cannula 100 according to an embodiment of the present disclosure.

FIG. 4 is a cross-sectional view of a first segment 110 of a cannula 100 according to various embodiments of the present disclosure when viewed from the side.

FIG. 5 is a side cross-sectional view of a cannula according to an embodiment of the present disclosure.

FIG. 6 is a diagram illustrating the circumference value range of a cannula according to an embodiment of the present disclosure.

FIG. 7 is a perspective view of an apparatus for injecting an implant for eye disease according to an embodiment of the present disclosure.

FIG. 8 shows an apparatus for injecting an implant for eye disease according to an embodiment of the present disclosure.

FIGS. 9A to 9C show a process of injecting an implant into eye by an apparatus for injecting an implant for eye disease according to an embodiment of the present disclosure.

FIGS. 9D and 9E shows an example of an implant for eye disease according to an embodiment of the present disclosure.

### BEST MODE

The terminology as used herein will be briefly described, and the present disclosure will be described in detail.

The terms as used herein are general terms selected as those being now used as widely as possible in consideration of functions in the present disclosure, but they may differ depending on the intention of those skilled in the art or the convention or the emergence of new technology. Additionally, in certain cases, there may be terms arbitrarily selected by the applicant, and in this case, the meaning will be described in the corresponding description part of the specification. Accordingly, the terms as used herein should be defined based on the meaning of the terms and the context throughout the specification, rather than simply the name of the terms.

The term “comprising” when used in this specification, specifies the presence of stated elements, but does not preclude the presence or addition of one or more other elements unless the context clearly indicates otherwise. Additionally, when an element is referred to as being “connected to” another element, it can be directly connected to the other element or intervening elements may be present.

Embodiments of the present disclosure will be described in sufficient detail for those skilled in the art to easily practice the present disclosure with reference to the accompanying drawings. However, the present disclosure may be embodied in many different forms and is not limited to the disclosed embodiments. Additionally, in the drawings, irrelevant elements to the description are omitted to clearly describe the present disclosure, and throughout the specification, similar reference signs are affixed to similar elements.

An apparatus for injecting an implant for eye disease according to embodiments of the present disclosure is designed to inject the tube-shaped implant into the eye to control the intraocular pressure by regulating the drainage of aqueous humor produced from the anterior chamber in front of the lens in the eye. The implant plays a role in preventing damage to the optic nerve by the increased intraocular pressure due to eye diseases. The implant for eye disease according to embodiments of the present disclosure may be used to treat many eye diseases that result in or from the increased intraocular pressure or mitigate symptoms.

The eye disease described herein may include glaucoma caused by the increased intraocular pressure, and the glaucoma may include congenital glaucoma, traumatic glaucoma, glaucoma suspect, ocular hypertension, primary open-angle glaucoma, normal tension glaucoma, capsular glaucoma with pseudoexfoliation of lens, chronic simple glaucoma, low-tension glaucoma, pigmentary glaucoma, primary angle-closure glaucoma, acute angle-closure glaucoma, chronic angle-closure glaucoma, intermittent angle-closure glaucoma, glaucoma caused by eye injury, glaucoma caused by eye inflammation, drug-induced glaucoma and neovascular glaucoma or secondary glaucoma caused by uveitis, but is not limited thereto.

The implant for eye disease may have a tubular shape that can be applied to Micro-Invasive Glaucoma Surgery (MIGS) such that one end of the tube is injected into the anterior chamber of the eye and the other end of the tube is injected into the conjunctival tissue or tenon tissue. In various embodiments of the present disclosure, the implant may come in various shapes, for example, linear, wedge, crisscross and cover-like shapes.

The implant for eye disease may be injected after a surgeon peels off the conjunctival tissue or tenon tissue of the eye, and after injected, may be placed in the eye by recovering the conjunctival tissue or tenon tissue. Alternatively, the implant for eye disease may be injected into the eye through the injection apparatus (an injector) according to an embodiment of the present disclosure, and thus part of the implant for eye disease may be injected into the anterior chamber of the eye. Here, the injection apparatus may be designed to inject the implant into the eye by pushing the implant received therein by a hand or a mechanical external force.

Hereinafter, the present disclosure will be described in detail with reference to the accompanying drawings.

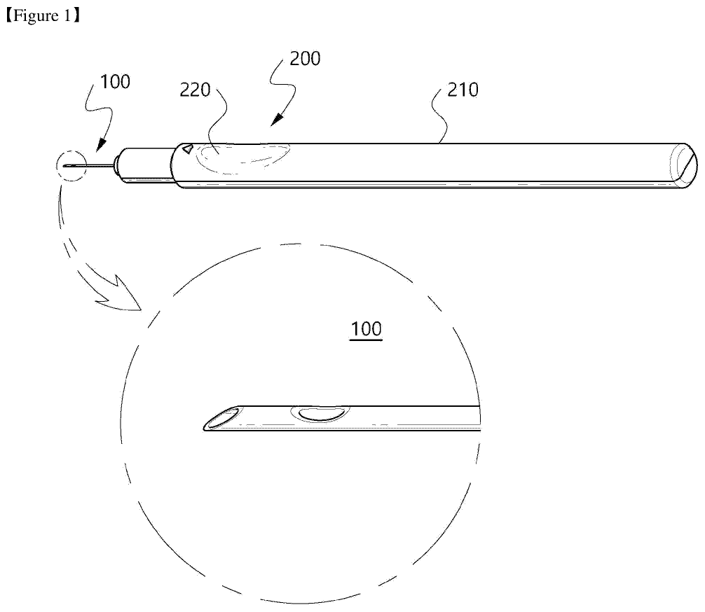

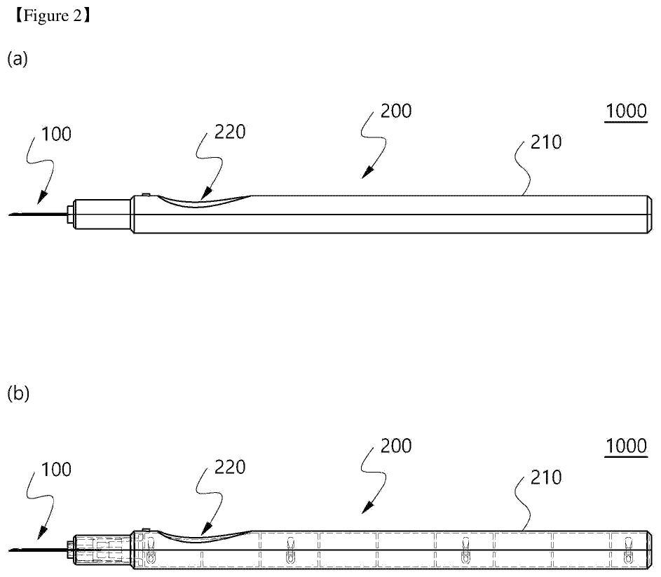

FIGS. 1 and 2 show an apparatus 1000 for injecting an implant for eye disease according to an embodiment of the present disclosure. Referring to FIG. 1, the apparatus 1000 for injecting an implant for eye disease may include a cannula 100 having an internal space. Additionally, the injection apparatus 1000 may further include a handle 200 which is connected to the cannula and allows a surgeon to grip.

The handle 200 may include a body 210 and a handle groove 220 which makes the surgeon's grip easy. Although not shown, in an example, the handle 200 may further include a gear structure (not shown) for moving the cannula 100 in the lengthwise direction or rotating the cannula 100. The gear structure allows the cannula 100 to move or rotate by a smaller amount than an amount of movement of the surgeon's hand motion (for example, forward/backward movement or rotation of a lever), thereby achieving precise cannula control. Meanwhile, the above-described linear or rotational movement may be simultaneously made through the gear of spiral structure.

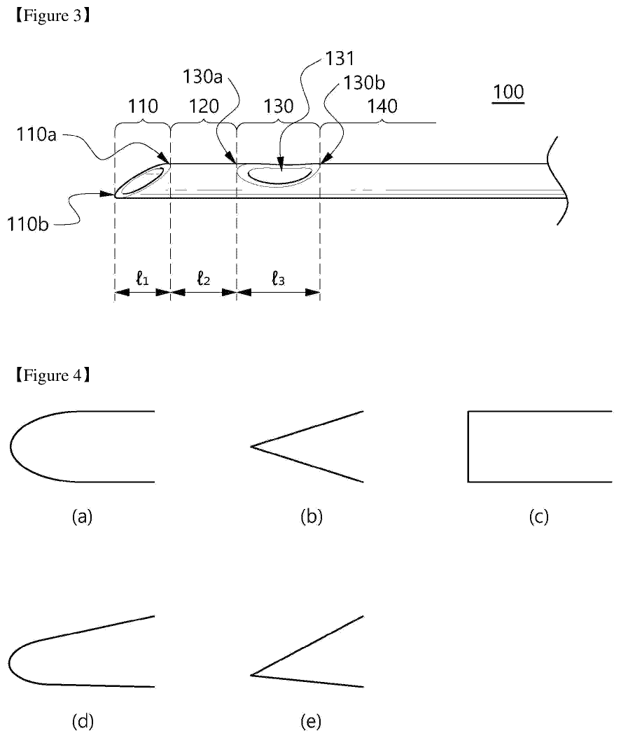

FIG. 3 shows the cannula 100 according to an embodiment of the present disclosure. Referring to FIG. 3, the cannula 100 includes a first segment 110 including one end 110b of the cannula, a second segment 120 connected to the first segment 110, and a third segment 130 connected to the second segment 120 and having an opening 131 for the implant on the side. In an example, when the end of the cannula has an inclined shape, the first segment refers to a length region including the inclined portion. Accordingly, when the end of the cannula has a surface of almost right angle, the first segment may be defined as a length that is as small as the length of the corresponding surface. That is, when the end of the cannula has an inclined surface, the first segment may be defined as a distance 11 on the lengthwise axis between the first end 110a and the second end 110b of the corresponding inclined surface. Accordingly, when the first end 110a and the second end 110b are on the same vertical line, the first segment may exist as a line having no volume.

Additionally, the third segment may be defined as a length portion in which the opening 131 for the implant is formed. Additionally, the second segment may be defined as a region between the first segment and the third segment.

In an example, the length of the second segment may be 1 mm or more. In the injection apparatus according to an embodiment of the present disclosure, the implant is injected into the eye with only part of the implant inserted into the cannula. In this instance, to properly fix the implant to the cannula, it is necessary to insert a predetermined length of the implant into the cannula. In this instance, part of the implant may be disposed at the second segment in the cannula. Accordingly, the length of the second segment is preferably at least 1 mm. The length of about 1 mm provides appropriate fixation when the implant is inserted into the cannula, thereby preventing the implant from slipping away from the cannula by the tissue in the eye when injecting the cannula into the eye. The length of the second segment is preferably 1 mm or more, but may be 3 mm or more.

Meanwhile, when injecting the cannula into the eye to inject the implant, in case that the injection depth is too deep, the cannula touches the tissue on the opposite side to the injection region, causing damage to the tissue in the eye. To prevent the damage, the sum of lengths of the first segment and the second segment of the cannula preferably has a predetermined length. In an example, the sum of lengths of the first segment and the second segment may be equal to or smaller than 10 mm. When the sum of lengths of the first segment and the second segment is larger than 10 mm, there is a risk that the tissue in the eye may be damaged in the process of injecting the implant into the eye and separating the implant in the eye.

That is, when the length of the second segment is too short, there is a high likelihood that the implant inserted into the cannula may slip off from the cannula due to the resistance of the eye tissue when injecting the cannula into the eye, so the length of the second segment is preferably 1 mm or more, and when the sum of lengths of the first segment and the second segment is larger than 10 mm, it is necessary to separate the cannula from the implant after injecting the cannula with the implant into the eye, but it is not easy to separate them and the tissue in the eye may be damaged.

Additionally, the cannula 100 may further include a fourth segment 140 connected to the third segment 130 and to the handle 200. The fourth segment 140 may be integrally formed with the handle 200 or coupled to the handle 200 with another element.

FIG. 4 is a cross-sectional view of the first segment 110 of the cannula 100 according to various embodiments of the present disclosure when viewed from the side. Referring to FIG. 4, the end of the first segment 110 of the cannula may have a curved shape (a), a pointed shape (b), a right angle cross section shape (c), a curved shape inclined to one side (d), or a pointed shape inclined to one side (e), but is not limited thereto. (b) of FIG. 4 shows the first segment 110 having a conic or cylindrical end that is truncated diagonally with respect to a point. That is, in an embodiment of the present disclosure, one end of the first segment 110 of the cannula may have at least one inclined surface. Additionally, in an example, one end of the cannula may be open. Referring to FIGS. 1 to 3, it is shown that the end of the first segment 110 of the cannula is open. When one end of the cannula is open, the surgeon may extract and see the implant through the opening and pull the implant again or push it into the cannula. In this case, the extracted implant may be pushed to the open surface or a location near the open surface, so the surgeon may use the opening at one end of the cannula to see the position of the implant in the cannula and fix the implant.

Referring back to FIG. 3, in the present disclosure, the first segment 110 of the cannula may refer to the segment where the inclined surface is formed, the third segment 130 may refer to the segment where the opening 131 for the implant is formed, and the second segment 120 may refer to the segment between the first segment 120 and the third segment 130.

Although FIG. 3 shows that the side of the second segment 120 is closed, in another embodiment, the second segment 120 may have at least one opening. However, the lengthwise width of the at least one opening in the second segment 120 may be smaller than the lengthwise width 13 of the opening 131 for the implant of the third segment 130. That is, the opening 131 for the implant is spaced a predetermined distance apart from one end of the cannula.

That is, when the length of the second segment is too small, when the cannula having the inserted implant is injected into the eye, there is a high likelihood that the implant will slip off from the cannula due to the resistance of the eye tissue, so the length of the second segment is preferably 1 mm or more, and when the sum of lengths of the first segment and the second segment is larger than 10 mm, it is necessary to separate the cannula from the implant after injecting the cannula having the inserted implant into the eye, but it is not easy to separate them.

To solve this problem, in the injection apparatus 1000 according to an embodiment of the present disclosure, the sum of lengths of the first segment and the second segment is defined as being equal to or less than 10 mm, so when the cannula is injected into the eye with part of the implant inserted in the cannula, it is possible to keep the implant in the injected state and easily separate the implant in the eye.

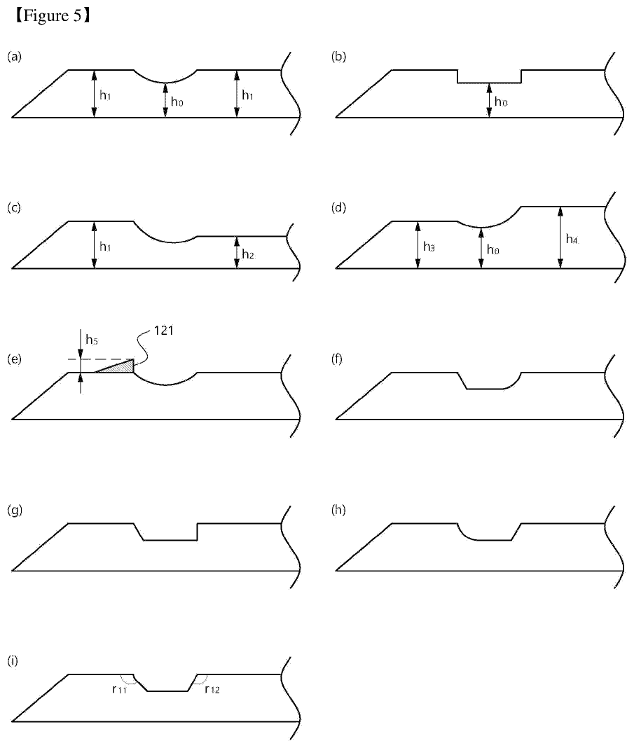

In an example, the opening for the implant may be defined by a first open end adjacent to the second segment and a second open end adjacent to the fourth segment in the lengthwise direction. In this instance, the first open end and the second open end may be different in height. In an example, the height of the first open end may be larger than the height of the second open end. Hereinafter, a change in height of the two ends in the lengthwise direction of the opening for the implant and its function will be described with reference to FIG. 5.

FIG. 5 is a side cross-sectional view of the cannula according to an embodiment of the present disclosure. Referring to (a) and (b) of FIG. 5, the height h₁ of the second segment may be equal to the height h₁ of the fourth segment and the height h₀ of the third segment may be smaller than the height h₁ of the second segment and the height h₁ of the fourth segment. That is, the opening for the implant may have a concave shape.

Additionally, as shown in (c) of FIG. 5, the height h₁ of the second segment may be larger than the height h₂ of the fourth segment. In this case, an angle between the implant inserted into the cannula and the cannula may be smaller than that of (a) or (b) of FIG. 5. Additionally, since the height of the second segment is larger than the height of the fourth segment, it is possible to reduce the resistance applied to the implant when injecting the cannula into the eye with part of the implant inserted into the cannula. That is, since the implant is injected in a state that it is more inclined toward the cannula, it is possible to reduce the injection area.

In another embodiment, on the contrary to (c) of FIG. 5, as shown in (d) of FIG. 5, the height of the fourth segment may be larger than the height of the second segment. In this case, an angle between the implant and the cannula may be higher. That is, part of the implant that is not inserted into the cannula may have a rising pose above the cannula. In this case, the surgeon may inject the cannula while he/she is easily holding the exposed part of the implant with forceps. Alternatively, even though the surgeon does not hold part of the implant with forceps, since a bent portion of the exposed part of the implant is pushed by the fourth segment, the fourth segment may perform a supporting function to prevent the implant from slipping off when injecting the cannula.

Additionally, in another embodiment, as shown in (e) of FIG. 5, the second segment may include a protrusion 121 adjacent to the opening for the implant. The protrusion may have a predetermined height h₅, and perform a function for reducing a force of the eye tissue pushing the exposed part of the implant when injecting the cannula into the eye. To this end, the protrusion may have a structure in which the height gradually increases in a direction from the first segment to the third segment.

Additionally, in another embodiment, referring to (f) to (i) of FIG. 5, the shape of the two ends in the lengthwise direction of the opening 131 for the implant may have different shapes such as an inclined plane, an inclined curved surface and an inclination difference. With varying shapes of the two ends in the lengthwise direction of the opening 131 for the implant, a suitable shape may be employed according to the shape of the implant to enhance the fixation when inserting into the cannula and injecting into the eye and make it easy to separate in the eye.

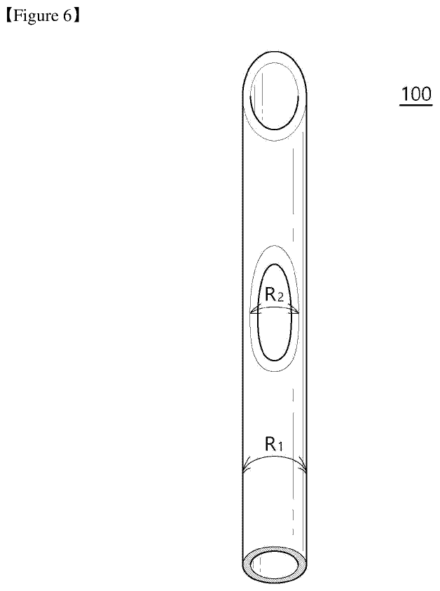

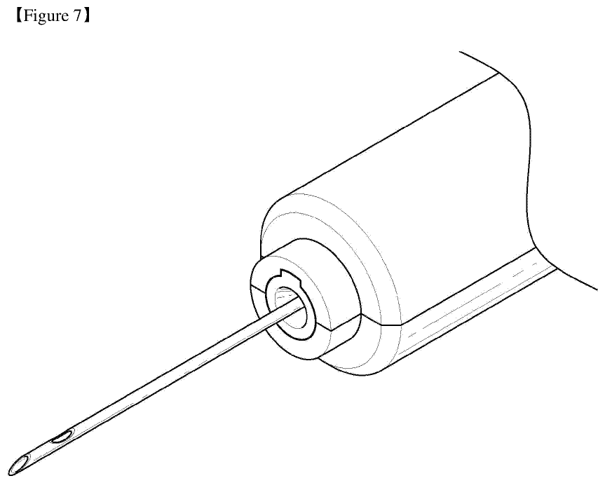

FIG. 6 is a diagram illustrating the circumference value range of the cannula according to an embodiment of the present disclosure. Referring to FIG. 6, the circumference R₂ in a second direction (i.e., a direction of rotation with respect to the lengthwise direction) of the opening for the implant may be equal to or less than 40% of the total circumference R₁ of the third segment. That is, preferably, 100*R₂/R₁ may be less than 40. When the R₂ value is too large, there is a risk that the area of the third segment 130 except the opening 131 for the implant will be broken or easily damaged by the resistance of the tissue in the eye when injecting the cannula. Accordingly, to prevent this problem, preferably, the circumference value of the opening for the implant does not exceed 40% of the total circumference.

In another example, the depth of the opening for the implant may be 40% of the outer diameter of the cannula. As shown below, the depth (i.e., the height of the empty region) in the vertical direction of the opening for the implant may be 40% of the outer diameter of the cannula.

However, this description is provided for illustration purposes, and the above-described ratio may be differently applied depending on the material of the cannula. The description of the ratio of the opening made with reference to FIG. 6 is intended to describe that it is necessary to appropriately adjust the area of the opening for the implant applied to the cannula depending on the material and structure of the implant and the material and structure of the cannula.

FIG. 7 is a perspective view of the apparatus for injecting an implant for eye disease according to an embodiment of the present disclosure. The surgeon may prepare the injection apparatus shown in FIG. 7, insert the implant for eye disease into the injection apparatus and inject it into the eye. Alternatively, the implant may be fabricated and packaged such that part of the implant is injected into the injection apparatus and then delivered to the surgeon.

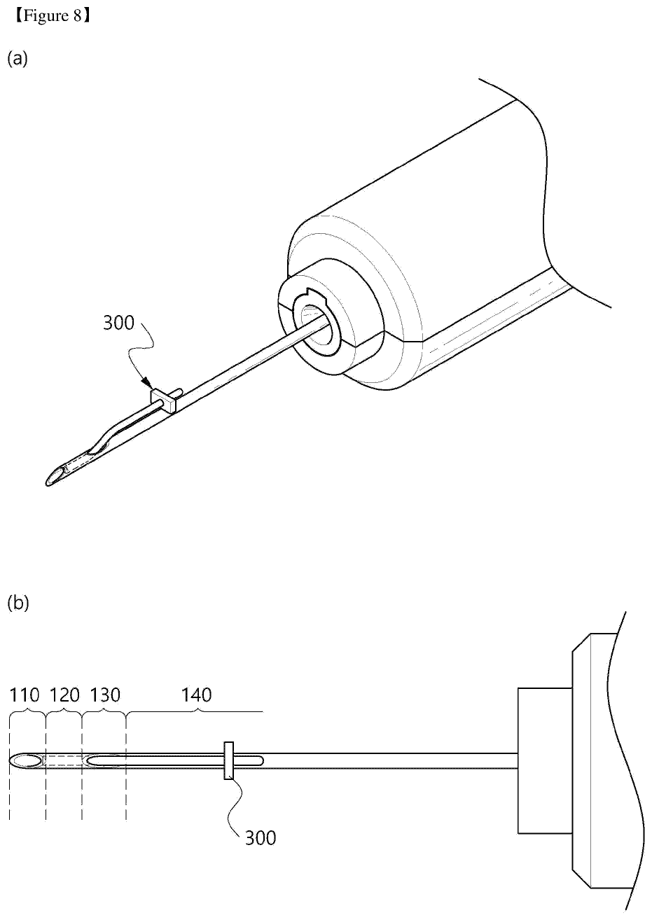

FIG. 8 shows the apparatus for injecting an implant for eye disease according to an embodiment of the present disclosure. Referring to FIG. 8, the apparatus 1000 for injecting an implant for eye disease may further include the implant 300 for eye disease. The implant for eye disease may be tubular in shape, and at least part of the implant for eye disease may be disposed in the second segment of the cannula. To place part of the implant in the second segment of the cannula, the implant may be inserted toward one end through the opening 131 for the implant, or may be inserted into one end such that part of the implant is extended through the opening 131 for the implant.

In an example, the implant 300 for eye disease may be in the shape of a tube that is extended in the lengthwise direction and has the inner diameter.

In an embodiment, the implant may be made of at least one of silicone, polytetrafluoroethylene (PTFE), polycarbonate, polyurethane, polyethylene, polypropylene, polyimide, poly(methyl methacrylate (PMMA), poly(styrene-b-isobutylene-b-styrene), polyethersulfone, gelatin, stainless steel, titanium or nitinol but is not limited thereto.

In an embodiment, to prevent damage to the corneal endothelium in the eye, the implant may form a curve having a predetermined curvature. Due to different eye sizes in each patient and the implant injection skills, the front end of the implant may poke the cornea in the anterior chamber of the eye, causing damage to the cornea in the process of injecting the implant into the anterior chamber of the eye, and the damage of the cornea may cause complications such as corneal dysfunction that may require a cornea transplant afterwards.

In an embodiment, the implant may be fabricated in bent shape with the predetermined curvature corresponding to the curvature of the eye surface to naturally form a curve in the process of injecting the implant into the anterior chamber of the eye. Alternatively, even in case that the implant is linear in shape, the implant having no curvature may be used by use of a material having sufficient elasticity or flexibility. Additionally, although FIG. 8 shows that a portion of the part of the implant that is not injected into the cannula is extended in a direction that is different from the lengthwise direction, this is provided for illustration purposes, and the implant may be extended in only one direction and may have any other structure.

Referring to FIG. 8, only part of the implant 300 for eye disease is inserted into the cannula toward the first segment (one end) through the opening for the implant. That is, in the present disclosure, only part of the implant 300 for eye disease is inserted into the cannula of the injection apparatus, and the implant 300 for eye disease is not completely inserted into the injection apparatus, or the injection apparatus is not injected into the eye in the inserted state into the implant. When inserting the implant for eye disease into the injection apparatus, and then injecting the injection apparatus into the eye and extracting the implant from the injection apparatus, the separation of the injection apparatus and the implant increases the movement range of the injection apparatus in the eye, resulting in increased eye tissue damage range. Additionally, to implement a separate mechanism for separating the implant inserted into the injection apparatus, the manufacturing cost rises due to the complicated structure of the injection apparatus. Additionally, the method for inserting the injection apparatus into the implant cannot be applied to micro-scale implants such as MIGS devices.

The apparatus 1000 for injecting an implant for eye disease according to an embodiment of the present disclosure inserts at least part of the implant 300 into the cannula and then injects the cannula into the eye, thereby providing the following advantages: 1) restricting the damage range of the eye surface tissue to some of the cross-sectional thickness of the cannula+the cross-sectional thickness of the implant in the process of injecting the cannula into the eye, and 2) separating the implant and the cannula in the eye in a straightforward manner.

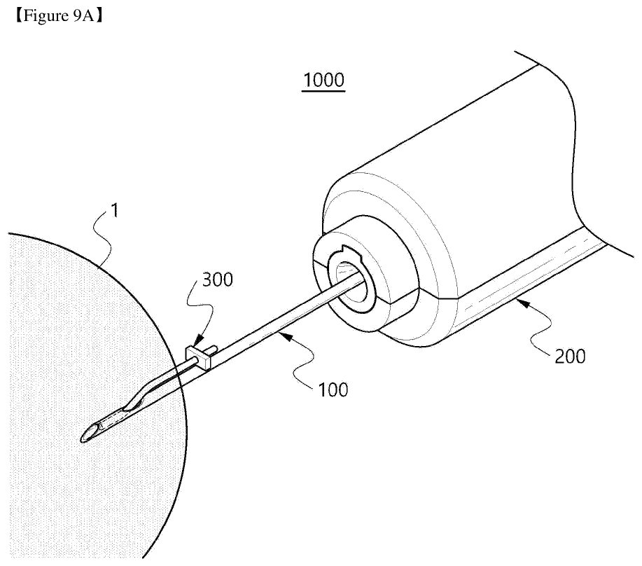

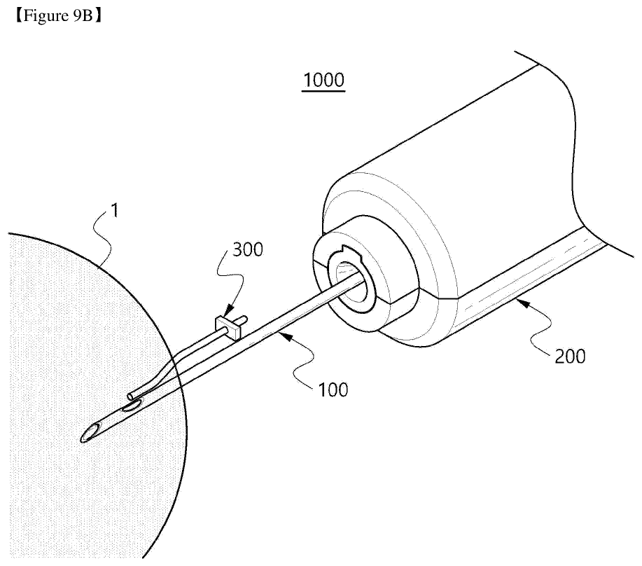

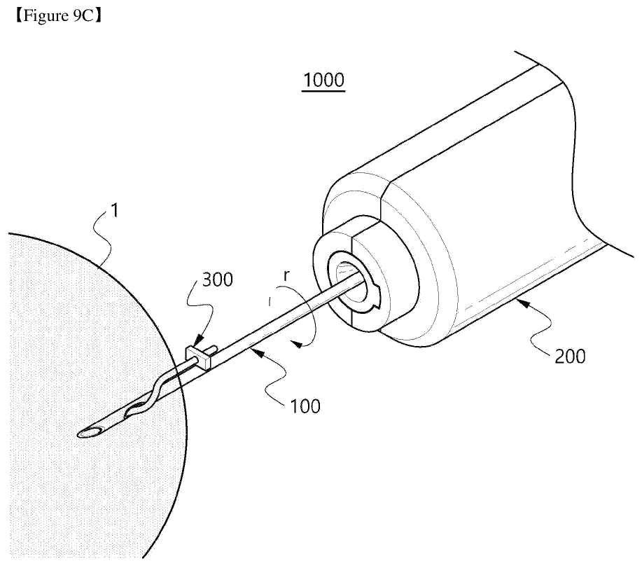

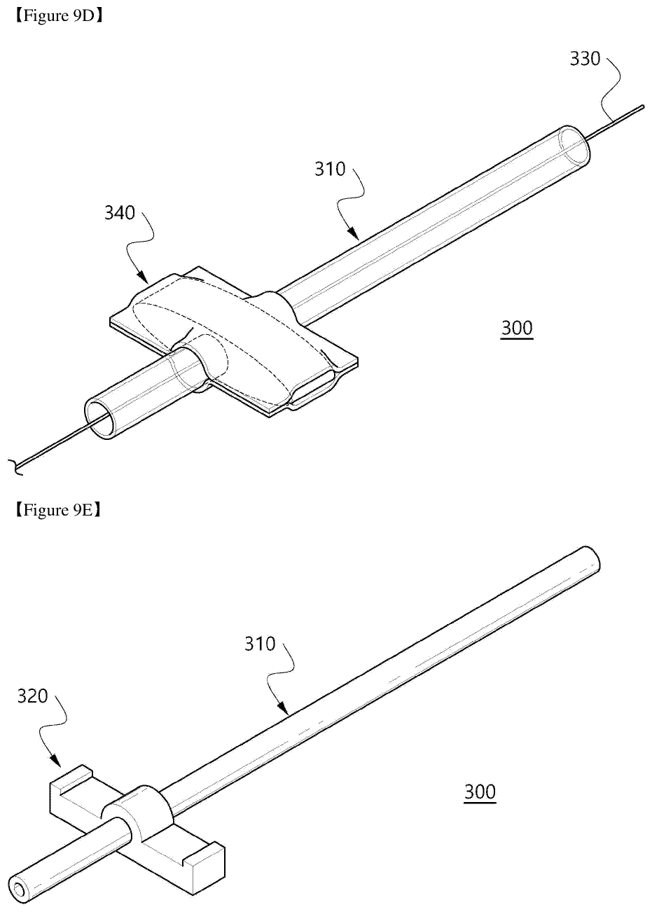

FIGS. 9A to 9C show the process of injecting the implant into the eye by the apparatus for injecting an implant for eye disease according to an embodiment of the present disclosure. Referring to FIG. 9A, the surgeon may inject the cannula into the eye 1 with part of the implant 300 inserted through the opening 131 for the implant as shown in FIG. 8. The surgeon may open the conjunctiva near the corneal limbus with surgical scissors, or subsequently, may make a scleral flap according to the scleral condition. Here, the scleral flap may have a square or trapezoidal shape having the size of approximately 3*3 mm and the depth may be about ±50% of the scleral thickness, but the present disclosure is not limited thereto.

The surgeon may inject the cannula into the eye with the implant inserted (loaded) into the cannula. For example, the surgeon may push the cannula into a location at least 1 to 2 mm away from the corneal limbus. In this instance, the implant injected into the anterior chamber may be disposed farthest away from the cornea.

When the cannula 100 is injected into the eye 1, the implant 300 inserted into the cannula is injected into the eye together. In this instance, the surface of the eye is open as wide as the cross-sectional area of the non-injected part of the implant and the cross-sectional area of the cannula. The implant 300 is made of a very flexible material, and comes into close contact with the side of the cannula due to the resistance of the tissue in the eye when injecting the cannula. Accordingly, it is possible to minimize the open area of the eye surface.

In an example, when the implant 300 is placed at a necessary location in the eye 1, the surgeon needs to fix the implant to the corresponding location and take out the cannula 100 from the eye.

To this end, as shown in FIG. 9B, the surgeon may rotate one end of the cannula 100 in a predetermined direction (opposite the direction in which the opening for the implant faces) to separate the implant from the cannula. That is, the surgeon may separate the implant through the opening for the implant by rotating the cannula 100 in the pitch direction. Alternatively, the surgeon may easily separate the implant by moving the cannula forward a predetermined distance at the same time with rotating the cannula in the pitch direction. In an example, the cannula preferably moves forward about 1 mm, but the present disclosure is not limited thereto.

To provide an explanation of the separation, since the first or second segment of the cannula is disposed in the anterior chamber and the fourth segment is disposed on the eye surface or outside of the eye, the fourth segment is supported by the eye surface tissue (the surface tissue tightens the cannula and the implant), and when the surgeon rotates the cannula in the pitch direction based on the support, the implant disposed at the anterior chamber may be easily separated from the opening for the implant.

In another example, when the implant in the eye reaches a predetermined location, the surgeon rotates the cannula in the roll direction, not in the pitch direction, to separate the implant from the cannula. Referring to FIG. 9C, as the surgeon rotates the cannula 100 in the roll direction, the implant is separated from the opening for the implant. When the surgeon rotates the cannula in the roll direction, in the similar way to the pitch direction rotation, the surgeon may rotate the cannula in the roll direction together with moving the cannula forward by the predetermined length (about 1 mm). The forward movement makes it easier to separate the implant. Additionally, in the case of the roll rotation, not pitch rotation, since the cannula is rotated as it is, when compared with the pitch rotation, it is possible to prevent the further expansion of the range of the eye surface tissue that is already wide open.

FIGS. 9D and 9E show an example of the implant 300 for eye disease according to an embodiment of the present disclosure. In an example, the implant for eye disease may include a body 310 and a wing 320 protruding in a direction that is different from the lengthwise direction of the body 310. FIGS. 9A to 9D show the wing 320. The wing 320 may be perpendicular to the lengthwise direction of the body and may be inclined at a predetermined angle. Additionally, the wing 320 may be extended in a direction, and have, on the side, an additional protruding portion that protrudes in an opposite direction (see FIG. 9D). The wing may prevent the implant injected into the eye from moving deeper into the eye.

In an example, the implant 300 for eye disease may include a core 330 in the body. The core 330 may perform a function of preventing the rapid drainage of aqueous humor immediately after the injection of the implant.

Additionally, referring to FIG. 9E, the implant 300 for eye disease may further include a matrix that covers the implant at least in part. The matrix 340 may perform the identical or similar function to the wing. That is, the matrix 340 may prevent the implant from moving deeper into the eye. Additionally, a drug for eye disease may be loaded in the matrix 340. For example, the drug for eye disease may be gradually released in the eye after it is impregnated into the matrix or injected into the internal space of the matrix.

In an embodiment, the drug that may be loaded in the matrix may include, for example, an anti-fibrotic drug such as dexamethasone, mitomycin-C (MMC), 5-fluorouracil (5-FU), triamcinolone (TA), an anti-Vascular Endothelial Growth Factor (anti-VEGF) drug and a Transforming Growth Factor (TGF)-beta inhibitor, but is not limited thereto. For example, in addition to the anti-fibrotic drug, an intraocular pressure lowering drug such as prostaglandin, Cephalosporin based antibiotics such as levofloxacin, moxifloxacin and ceftazidime or any other type of antibiotics or any other type of drug may be loaded in the matrix according to the purpose of applying the drug to the matrix. Additionally, the matrix may be made of a material that does not immediately decompose in the human body to allow the drug release for a sufficient length of time, and does not expand to such a large extent that the drug causes a feeling of irritation in the eye. In an embodiment, the matrix may include a polymer of a layer structure that can form a storage space for entrapping the drug.

In an example, the matrix 340 may be made of at least one of poly(acrylic acid), polyacrylamide, poly(sulfopropyl acrylate), poly(2-hydroxyethyl methacrylate), poly(vinyl alcohol), silicone, polyurethane, collagen, gelatin, hyaluronic acid, poly(aspartic acid), alginate, hydroxypropyl cellulose, hydroxypropyl methylcellulose acetate succinate or chitosan, but is not limited thereto.

In an embodiment, the matrix 340 may be a material having a double layer structure formed by at least two different materials. In this instance, a first material of which the matrix 340 is made may be a material which has biocompatibility but does not biodegrade immediately in the eye and has mechanical properties for forming the skeleton of the matrix 340. Additionally, a second material or another material which makes up the matrix 340 together with the first material may be a material that has hydrophilicity and can temporarily absorb a water-soluble drug when it is bonded to the first material. When the matrix 340 is made of a complex of the first material and the second material as in this embodiment, it is possible to easily impregnate the drug suitable for the purpose into the matrix component 12 while maintaining the mechanical strength of the matrix 340. For example, the matrix 340 may be made of a PU-PEG blend in a sheet shape.

In an embodiment, the first material may include at least one of silicone, polyethylene vinyl acetate, polyvinyl acetate, polycarbonate, polyvinyl chloride, polyurethane, PMMA, poly(butyl methacrylate), polyamide, polyethylene, polypropylene, polyethylene terephthalate (PET), glycol-modified PTE, PTFE, polyhydroxyalkanoates, parylene, polyether ether ketone, polyimide, epoxy based resin, for example, SU-8, poly(vinylidene fluoride), polyether block amides, 3-[tris(trimethylsiloxy)silyl]propyl methacrylate, poly(styrene-isobutylene-styrene) copolymers, cellulose acetate, poly((N-vinylpyrrolidone)-block-poly(vinyl acetate) or ethyl cellulose, but is not limited thereto.

Additionally, in an embodiment, the second material may include at least one of poly(ethylene glycol) (PEG), PEG derivatives, poly(ethylene oxide), poly(propylene oxide), polyvinylalcohol, polyvinyl pyrrolidone, polyethersulfone, polyamide, polyacrylamide, polyglycolic acid, polyacrylic acid, glucuronic acid, hexuronic acid, hyaluronic acid, poly(aspartic acid), alginate, polyorthoester, 2-hydroxyethyl methacrylate, collagen, gelatin, hydroxypropyl cellulose or chitosan, but is not limited thereto.

The foregoing description of the present disclosure is provided for illustration purposes, and those skilled in the art will understand that the present disclosure can be easily modified in any other form without changing the technical spirit or essential features of the present disclosure. Therefore, it should be understood that the embodiments described hereinabove are provided by way of illustration in all aspects, but not intended to limiting. For example, each element described in singular form may be distributed, and likewise, the distributed elements may be combined.

The scope of the present disclosure is defined by the appended claims rather than the foregoing description, and it should be interpreted that all modifications or changes from the meaning and scope of the appended claims and the equivalents thereto fall in the scope of the present disclosure.

### INDUSTRIAL APPLICABILITY

According to the apparatus for injecting an implant for eye in accordance with an embodiment of the present disclosure, it is possible to minimize the eye tissue damage range when injecting the implant for eye disease into the anterior chamber of the eye, and keep the size of the internal structure of the injection apparatus small since part of the implant that is not injected into the anterior chamber is not inserted into the injection apparatus and is disposed outside of the injection apparatus, and thus it is industrially applicable.

1. An apparatus for injecting an implant for eye disease, comprising:
a cannula having an internal space,
wherein the cannula includes:
a first segment including an end of the cannula;
a second segment connected to the first segment; and
a third segment connected to the second segment, and including an opening for the implant on a side.

2. The apparatus for injecting an implant for eye disease according to claim 1, wherein a length of the second segment is equal to or larger than 1 mm.

3. The apparatus for injecting an implant for eye disease according to claim 1, wherein a sum of lengths of the first segment and the second segment is equal to or smaller than 10 mm.

4. The apparatus for injecting an implant for eye disease according to claim 1, wherein the opening for the implant has different heights of a first open end and a second open end,
wherein the first open end is adjacent to the second segment, and
wherein the second open end is opposite the first open end.

5. The apparatus for injecting an implant for eye disease according to claim 4, wherein each of the first open end and the second open end has any one shape of a sloped surface inclined inward the cannula, an inclined curved surface, a vertically curved surface and a vertically sloped surface.

6. The apparatus for injecting an implant for eye disease according to claim 5, wherein angles at which the first open end and the second open end are inclined inward the cannula are different from each other.

7. The apparatus for injecting an implant for eye disease according to claim 4, wherein the height of the first open end is larger than the height of the second open end.

8. The apparatus for injecting an implant for eye disease according to claim 1, wherein the end of the cannula is open.

9. The apparatus for injecting an implant for eye disease according to claim 1, wherein the second segment includes a protrusion disposed adjacent to the opening for the implant.

10. The apparatus for injecting an implant for eye disease according to claim 1, further comprising:
the implant for eye disease,
wherein a body of the implant for eye disease is tubular in shape, and
wherein at least part of the implant for eye disease is disposed in the second segment.

11. The apparatus for injecting an implant for eye disease according to claim 10, wherein part of the implant for eye disease is inserted into the cannula toward the first segment through the opening for the implant.

12. The apparatus for injecting an implant for eye disease according to claim 10, wherein the implant for eye disease includes a core therein.

13. The apparatus for injecting an implant for eye disease according to claim 10, wherein the implant for eye disease includes a wing protruding in a direction that is different from a lengthwise direction.

14. The apparatus for injecting an implant for eye disease according to claim 10, wherein the implant for eye disease further includes a matrix which covers the implant at least in part.

15. The apparatus for injecting an implant for eye disease according to claim 14, wherein the matrix is impregnated or impregnable with a solution for eye disease.
